(19) United States
(12) Patent Application Publication
(10) Pub. No.: US 2023/0349892 A1
(43) Pub. Date: Nov. 2, 2023

(54) HIGH SENSITIVITY IMMUNOASSAY

(71) Applicant: Bio-Rad Laboratories, Inc., Hercules, CA (US)

(72) Inventors: William STRONG, El Cerrito, CA (US); Eli HEFNER, Fairfield, CA (US); Carl MARLOWE, San Francisco, CA (US)

(21) Appl. No.: 18/139,829

(22) Filed: Apr. 26, 2023

Related U.S. Application Data
(60) Provisional application No. 63/335,379, filed on Apr. 27, 2022.

(51) Int. Cl. G01N 33/543; G01N 33/58

(52) U.S. Cl. CPC G01N 33/543; G01N 33/582; G01N 2458/10

(57) ABSTRACT

The disclosure provides a highly sensitive assay to detect the presence of a target analyte in a sample.

### CROSS-REFERENCE TO RELATED APPLICATIONS

This application claims priority to U.S. Patent Application No. 63/335,379, filed Apr. 27, 2022, which is incorporated herein by reference in its entirety.

### BACKGROUND OF THE INVENTION

High sensitivity immune-detection assays often employ matched pairs of specific high-affinity antibodies oriented in a sandwich format. One antibody serves as an analyte capture molecule and the second serves as a detection molecule that can be measured either directly (e.g. fluorescently labeled) or indirectly via the binding of a labeled reporter directed towards the detection molecule or a tag linked to the detection molecule (e.g., streptavidin-fluor or streptavidin-enzyme binding to a biotin-tagged detection antibody molecule). Examples of high sensitivity immunoassays include those performed on the Luminex platform and Quanterix platforms.

An additional example of a highly sensitive immune assay is SiMREPS, which is based on single molecule detection using TIRF microscopy (see, e.g., US2019/0339266; Johnson-Buck et al., Nat Biotechnol. 33(7):730-732, 2015). SiMREPS provide the ability to resolve non-specific and specific target binding interactions, which leads to improved sensitivity by reducing/eliminating background. The SiMREPS technology employs detection molecules, e.g., antibody Fabs that have fast on-rate and fast off-rate kinetics, such that the temporal measure of repetitive detection probe binding can be used to distinguish specific from non-specific binding.

### BRIEF SUMMARY OF THE INVENTION

The present disclosure provides an assay employing both high affinity binding agents that target an analyte of interest and binding agents having fast off rates and relatively lower affinity for binding to the target analyte for detection and quantification of the analyte in a sample. Thus, in one aspect, the disclosure provides a method of quantifying a target analyte, the method comprising incubating (i) a solution comprising a complex that comprises a target analyte bound to a detection agent, wherein the detection agent binds with high affinity to the target analyte and is labeled with a detectable label; and (ii) a population of capture agents immobilized to a solid support in a channel or compartment; wherein the capture agents specifically bind the analyte and have a high off-rate of binding to the target analyte, and the analyte:detection agent complex migrates through the channel or compartment; spatially resolving the complex from other molecules in the solution based on rate of migration of the complex across the solid support relative to unbound detection agent to provide an eluate comprising the complex substantially free of unbound detection agent; and quantifying the amount of detection agent complexed with analyte, thereby determining the amount of the target analyte present in the solution. In a further aspect, the population of capture agents is immobilized in a discrete zone on a solid support, e.g., a membrane. In some embodiments, the capture agents are Fabs or aptamers. In some embodiments, the high affinity detection agent is an aptamer, antibody, or ligand. In some embodiments, the detectable label is specific for the analyte. In some embodiments, the detectable label is an oligonucleotide. In some embodiments, the oligonucleotide comprises a primer binding site or comprises a sequence that targets a primer-binding site of a detection oligonucleotide that is detected in an amplification reaction. In some embodiments, the oligonucleotide comprises an analyte identification region and/or a sample identification region. In some embodiments, the step of quantifying comprises amplifying a target region of the oligonucleotide to obtain an amplicon. In some embodiments, the step of amplifying comprises a PCR, such as quantitative PCR (qPCR) or digital PCR (dPCR). In some embodiments, quantifying comprises sequencing a region of the oligonucleotide specific for the analyte to determine the amount of oligonucleotide present in the detection agent-analyte complex. In some embodiments, the complex comprises a fluorescent label. In some embodiments, quantifying comprises detecting the level of fluorescent signal generated by the fluorescent label. In some embodiments, the channel or compartment is a capillary. In some embodiments, the solid support is a plurality of beads present in the channel or compartment. In some embodiments, the channel or compartment contains a solid support comprised of a polymer. In some embodiments, pressure or an electric field is applied to the solution as it flows through the channel or compartment. In some embodiments, the channel or compartment is a channel of a microfluidic device. In some embodiments, the channel is a capillary present in a microwell. In some embodiments, the solid support is a membrane or wicking matrix and the solution comprising the complex that comprises a target analyte bound to a detection agent flows through the membrane or wicking matrix by lateral flow.

In a further aspect, described herein is a kit comprising a capture agent as described herein bound to a solid support, and a high affinity antibody. In some embodiments, the solid support is a microbead or microparticle. In some embodiments, the solid support is a membrane or wicking material.

### BRIEF DESCRIPTION OF THE DRAWINGS

FIG. 1 provides an overview of an illustrative embodiment of an immunoassay using a binding agent having fast-off rate and on-rate kinetics as a capture agent linked to a solid support.

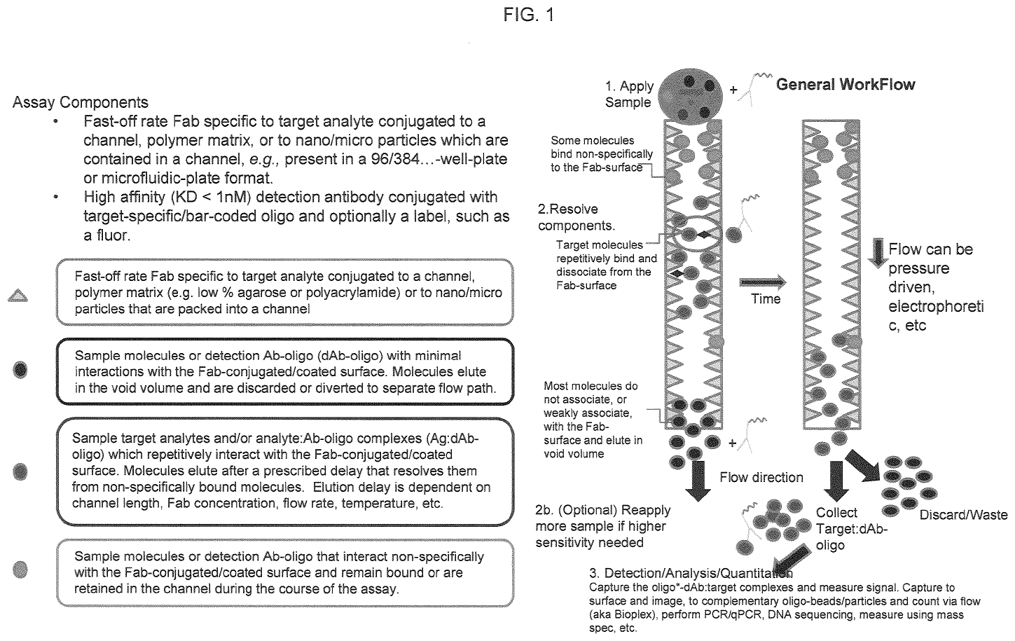

FIG. 2 depicts an illustrative assay.

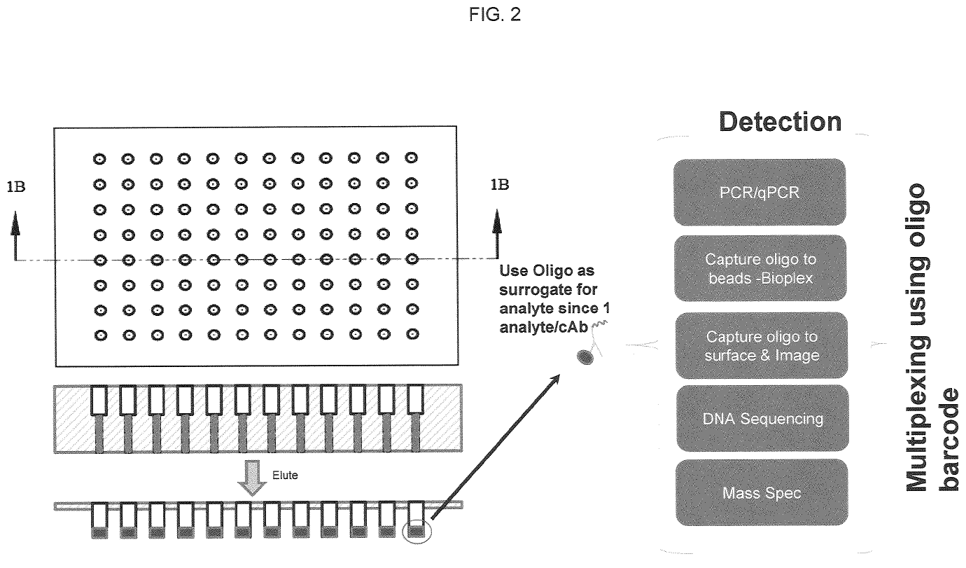

FIG. 3 depicts a top view of an illustrative lateral flow assay.

FIG. 4 depicts a side view of an illustrative lateral flow assay

FIG. 5 depicts an example of a “double Y” lateral flow assay.

### DETAILED DESCRIPTION OF THE INVENTION

Terminology

The term “a” or “an” is intended to mean “one or more.” The term “comprise,” and variations thereof such as “comprises” and “comprising,” when preceding the recitation of a step or an element, are intended to mean that the addition of further steps or elements is optional and not excluded. Any methods, devices and materials similar or equivalent to those described herein can be used in the practice of this invention. The following definitions are provided to facilitate understanding of certain terms used frequently herein and are not meant to limit the scope of the present disclosure.

The term “about” refers to the recited number and any value within 10% of the recited number.

The term “binding agent” as used herein refers to a binding molecule that specifically binds to a target molecule, e.g., an analyte to be detected in a sample. Examples of binding agents include, but are not limited to, antibodies or antibody fragments, aptamers, haptens, ligands, scaffold-based polypeptides, or other molecules that specifically bind to an analyte of interest.

As used herein, “affinity” is used to describe in general the strength of the interaction of a binding agent with an analyte, or with a molecule that interacts with the binding agent. The binding agent may have single or multiple binding sites to the analyte, or to the alternative molecule that interacts with the binding agent. In the present disclosure, the affinity of a binding agent and an analyte to which it binds is represented by the equilibrium dissociation constant (K_{D}), which is a ratio of the off-rate of the binding agent from the analyte (k_{d} or k_{off}) to the on-rate of the binding agent binding to analyte (kₐ or kₒₙ), i.e., K_{D}=k_{off}/kₒₙ. K_{D} is inversely related to the binding affinity, for example of an antibody to an antigen, where the smaller the K_{D} value, the greater the affinity of the binding agent for its target analyte.

The term “antibody” herein is used in the broadest sense and encompasses various antibody structures, including, but not limited to, monoclonal antibodies, polyclonal antibodies, multispecific antibodies, and antibody fragments, so long as they exhibit the desired antigen-binding activity. An antibody fragment refers to a portion of an intact antibody that binds a target antigen. Examples of antibody fragments include, but are not limited to Fab, Fab′, Fab′-SH, F(ab′)2, and Fv; diabodies; linear antibodies; single-chain antibodies such as single-chain variable fragments (scFv), fusions of light and/or heavy-chain antigen-binding domains with or without a linker (and optionally in tandem); and monospecific or multispecific antigen-binding molecules formed from antibody fragment, e.g., antibodies constructed from multiple variable domains that lack Fc regions.

“Protein” or “polypeptide” are used herein interchangeably to refer to a polymer of amino acid residues. As used herein, “protein” or “polypeptide” includes amino acid polymers in which one or more amino acid residues is a non-naturally occurring analog of a corresponding naturally occurring amino acid.

A “polynucleotide” or “nucleic acid” includes any form of RNA or DNA, including, for example, genomic DNA; complementary DNA (cDNA); DNA molecules produced by amplification; or synthetically produced DNA or RNA molecules. The terms include chimeric molecules and molecules comprising non-standard bases, modifications, or nucleotide analogs. For example, an oligonucleotide may contain naturally occurring nucleotides and/or analogs thereof. Polynucleotides may be single-stranded or double-stranded, or have both single-stranded and double-stranded regions (e.g. hairpins).

The term “analyte” as used herein refers to any molecule that can be detected in a sample by a binding agent that specifically binds to the molecule. Analytes include, but are not limited to, polypeptides, nucleic acids, carbohydrates, lipids, hormones, or other molecules of interest present in a sample, including macromolecules or complexes.

The terms “label” and “detectable label” interchangeably refer to a composition detectable by spectroscopic, photochemical, biochemical, immunochemical, chemical, or other physical means. For example, useful labels include fluorescent dyes (fluorophores), fluorescent quenchers, luminescent agents, electron-dense reagents, enzymes (e.g., as commonly used in an ELISA), biotin, digoxigenin, ³²P and other isotopes, haptens, proteins, nucleic acids, or other substances which may be made detectable, e.g., by incorporating a label into an oligonucleotide, peptide, or antibody specifically reactive with a target molecule. The term includes combinations of single labeling agents, e.g., a combination of fluorophores that provides a unique detectable signature, e.g., at a particular wavelength or combination of wavelengths. A “detectable label” as used herein includes reference to an oligonucleotide, which can be detected by PCR, sequencing or other biochemical reactions.

A molecule that is “linked” to a label (e.g., a labeled antibody) is one that is bound, either covalently, through a linker or a chemical bond, or noncovalently, through ionic, van der Waals, electrostatic, or hydrogen bonds to a label such that the presence of the molecule may be detected by detecting the presence of the bound label.

### Introduction

The present disclosure employs both high affinity binding agents that target an analyte of interest and binding agents having fast off rates and relatively lower affinity for binding to the target analyte for detecting or quantifying the analyte of interest in a sample. In the present disclosure, a fast off-rate binding molecule (e.g., a Fab fragment or aptamer specific to target analyte) is used for analyte capture, while the high-affinity agent, e.g., a high-affinity antibody or aptamer, is used for detection of the agent, for example by detecting a signal from a label attached to or otherwise incorporated into the high affinity binding agent and/or by amplification and/or sequencing using an oligonucleotide attached to the high affinity binding agent. For purposes of this disclosure, the binding agent that binds to a target analyte of interest that has a fast off-rate is referred to as the “capture agent” and the high affinity binding agent that binds to the target analyte of interest is referred to as the “detection agent”. Thus, as used herein, the “capture” agent repeatedly binds to an dissociates from the analyte-detection agent complex.

In the present disclosure, a sample to be evaluated for the presence of analyte is incubated with an excess of detection agent to generate an analyte-detection agent complex when the target analyte is present in the sample. The analyte-detection agent complex is subsequently incubated with the capture agent linked to a solid support, for example, linked to beads or microparticles contained in a channel or compartment, or linked to a compartment/channel of a microfluidics device or a microwell, under conditions in which the analyte-detection complex migrates through the compartment/channel containing the immobilized capture agent. The capture agent may also be linked to or incorporated into a polymer matrix such as polyacrylamide, agarose, or other cross-linked, branched or linear polymer known to permit sieving of polypeptides and/or nucleic acids. Said polymer matrix may be contained in a channel or compartment. Samples can be applied to individual channels or compartments or in some embodiments the solid support can be a larger format with multiple wells or reservoirs for holding different samples in a spatially resolved manner.

In some embodiments, a capture agent may be directly linked to the surface of a compartment or channel having small dimensions (e.g., 2-5 microns), e.g., a microfluidic chamber. Capture agents can be attached to the surface using cross-linked or linear polymers that extend from the surface into the channel lumen. In such embodiments, small amounts of sample would be employed and the length of the separation to achieve adequate resolution of the bound and free detection agent is determined.

The target analyte in the analyte-detection complex binds to the capture agent, but due to the fast off-rate of the capture agent, quickly dissociates. The analyte-detection agent complex thus repeatedly binds to and dissociates from the capture agents, thereby slowing migration of the analyte-detection agent through the compartment/channel or matrix relative to unbound detection moiety. Pressure or an electric field is typically applied to the solution comprising analyte-detection agent to flow the analyte-detection agent through the compartment or matrix containing immobilized capture agent, although a force may also be generated by other mechanisms, e.g., centrifugal force. Reagents and methods of the present disclosure are further detailed below.

### Binding Agents

A binding agent can be any molecule that exhibits specific binding to an analyte of interest. In some embodiments, the binding agent comprises an antibody, or a binding fragment thereof, that specifically binds to the target analyte. In some embodiments, the binding agent comprises an aptamer that specifically binds to the target analyte. In some embodiments, the aptamer is a peptide aptamer. In other embodiments, the aptamer is a polynucleotide aptamer. In some embodiments, the binding moiety comprises a ligand that binds to a site on a target analyte.

As used herein, the term “specifically binds to,” as used with reference to an affinity agent, refers to an affinity agent (e.g., an antibody) that binds to an antigen with at least 5-fold greater affinity than to non-antigen molecules, e.g., 6-fold, 7-fold, 8-fold, 9-fold, 10-fold, 20-fold, 25-fold, 50-fold, 100-fold, 10³-fold, 10⁴-fold, 10⁵-fold, 10⁶-fold, 10⁷-fold, 10⁸-fold, 10⁹-fold, 10¹⁰-fold, 10¹¹-fold, 10¹²-fold, 10¹³-fold, 10¹⁴-fold, or 10¹⁵-fold greater affinity. For example, an affinity agent that specifically binds a particular antigen will typically bind the antigen with at least a 2-fold greater affinity than to a non-antigen molecule.

In some embodiments, the binding agent is an antibody. As noted above, the term “antibody” encompasses full-length antibody formats, e.g., IgG, and functional fragments of antibodies that bind the target antigen, including multimeric and monomeric forms. Accordingly, antibodies that can be employed include diabodies, triabodies, tetrameric forms, single domain antibodies and the like. A functional fragment can be a portion of an antibody such as a F(ab′)2, Fab′, Fab, Fv, or can be an engineered binding fragments, such as an scFV. In some embodiments, the binding moiety may be an antibody mimetic. Examples include fibronectin-scaffold based polypeptides such as adnectins, ankyrin repeat scaffolds such as DARPins, and lipocalin-scaffold based polypeptides such as anticalins, and affibodies (see, e.g., Engineered Protein Scaffolds as Next-Generation Therapeutics, Annual Review of Pharmacology and Toxicology 2020, Vol 60: 391-415).

In some embodiments, the binding agent is a ligand that binds to a specific site of a target cellular molecule, e.g., target protein analyte, and includes ligands for receptors, enzymes, or other proteins. The ligand may be a polypeptide molecule, small molecule, or any molecule that binds to a cognate cellular binding partner.

In some embodiments, the binding moiety can be a nucleic acid or peptide aptamer. Aptamers interact with their targets by recognizing a specific three-dimensional structure. Peptide aptamers are composed of a short variable peptide loop attached at both ends to a protein scaffold such as the bacterial protein thioredoxin-A. A peptide aptamer specific to a target of interest may be selected using any method known by the skilled person such as the yeast two-hybrid system, ribosome display, or phage display. Peptide aptamers may be produced by chemical synthesis or recombinantly produced.

In some embodiments, the aptamer is a nucleic acid aptamer. Nucleic acid aptamers are a class of small nucleic acid ligands that are composed of RNA or single-stranded DNA oligonucleotides folded into a three-dimensional structure that have high specificity and affinity for their targets. For example, Systematic Evolution of Ligands by Exponential enrichment (SELEX) technology can be used to obtain aptamers specific to a particular molecular target. Nucleic acid aptamers can be produced by chemical synthesis or in vitro transcription for RNA aptamers. Nucleic acid aptamers include DNA aptamers, RNA aptamers, XNA aptamers (nucleic acid aptamer comprising xeno nucleotides) and L-RNA aptamers.

### High Affinity Binding Agent

As described herein, a binding agent exhibiting high affinity binding to an analyte is contacted with a sample comprising the target analyte. In some embodiments, the binding agent is an antibody, e.g., a bivalent or multivalent antibody. In some embodiments, the binding agent is an aptamer or ligand that binds a target analyte, e.g., a receptor.

In typical embodiments, the high affinity binding agent binds to the target analyte with a K_{D} of about 1 nM or less, or in some embodiments, a K_{D} of 100 pM or 10 pM or less. K_{D} can be measured using any technique including, e.g., ELISA-based methods, as well as other biophysical methods, such as micro-scale thermophoresis (MST), surface plasmon resonance (SPR), and biolayer interferometry (BLI). By way of illustration, SPR can be used. SPR has several steps, for example an analyte is immobilized to the device surface and then interrogated with the binding agent, which forms a signal over time that can be used to calculate the binding on-rate (kₒₙ). The next step typically replaces the binding agent with the buffer solution and the rate of binding agent dissociation (off-rate, k_{off}) is measured as the signal returns to baseline. The K_{D} is calculated from the ratio of k_{off}/kₒₙ.

High affinity binding agents as employed in the methods described herein typically have a low off-rate such that the high affinity binding agent remains bound to the analyte during the course of the assay. For example, the off-rate for dissociation from the analyte of the high affinity detection agent can be 10-fold or 100-fold or 1000-fold or lower than the off-rate of the capture agents (see, e.g., Chang et al, J. Immunol. Methods 378:102-115, 2012).

The following Table illustrates capture agents with favorable K_{D} values and having fast association and fast dissociation kinetics for use in the present methods.

TABLE 1
Illustrative capture agent kinetics

| | | | | mean half-life |
|---|---|---|---|---|
| KD | units | kon (M−1s−1) | koff (s−1) | (~1/koff, sec) |
| 1 | uM | 1.00E+07 | 10.00 | 0.1 |
| 1 | uM | 1.00E+06 | 1.000 | 1 |
| 1 | uM | 1.00E+05 | 0.100 | 10 |
| 1 | uM | 1.00E+04 | 0.010 | 100 |
| 1 | uM | 1.00E+03 | 0.001 | 1,000 |
| 100 | nM | 1.00E+07 | 1.000 | 1 |
| 100 | nM | 1.00E+06 | 0.100 | 10 |
| 100 | nM | 1.00E+05 | 0.010 | 100 |
| 100 | nM | 1.00E+04 | 0.001 | 1,000 |
| 100 | nM | 1.00E+03 | 0.0001 | 10,000 |
| 10 | nM | 1.00E+07 | 0.100 | 10 |
| 10 | nM | 1.00E+06 | 0.0100 | 100 |
| 10 | nM | 1.00E+05 | 0.0010 | 1,000 |
| 10 | nM | 1.00E+04 | 0.0001 | 10,000 |
| 10 | nM | 1.00E+03 | 0.00001 | 100,000 |
| 1 | nM | 1.00E+07 | 0.0100 | 100 |
| 1 | nM | 1.00E+06 | 0.0010 | 1,000 |
| 1 | nM | 1.00E+05 | 0.0001 | 10,000 |
| 1 | nM | 1.00E+04 | 0.00001 | 100,000 |
| 1 | nM | 1.00E+03 | 0.000001 | 1,000,000 |

In some embodiments, detection agents are provided in molar excess relative to the amount of analyte to maximize formation of detection agent-analyte complexes. In some embodiments, detection agents are provided at concentrations that are in excess of about 1 pM to about 1 mM, most often about 0.1 nM to about 1 pM, or about 1 nM, 5 nM, 10 nM, 20 nM, 30 nM, 50 nM, 100 nM, 200 nM, 300 nM, 400 nM, 500 nM, or 1 μM relative to the concentration of analyte in a sample.

In some embodiments, the high affinity detection reagent, e.g., antibody, is conjugated with an oligonucleotide that is specific for a target being analyzed and/or contains a sample identification region. In some embodiments the oligonucleotide includes one or more labels, such as a fluorescent label, for detection purposes.

In some embodiments, an oligonucleotide comprises a primer binding site, or a region that hybridizes to a target primer binding site of a detection oligonucleotide to be amplified, that can be used in detecting and quantifying an analyte in a sample, e.g., using PCR, qPCR, and/or DNA sequencing. In some embodiments, the oligonucleotide further comprises an analyte identification region specific for the analyte, for example to identify each analyte detected in a multiplex reaction by sequence analysis. In some embodiments, the oligonucleotide further comprises a sample identification region. In some embodiments, the oligonucleotide further comprises a region or linker that allows for removal of the oligonucleotide from the high affinity detection reagent for subsequent analysis or identification.

In some embodiments, qPCR or digital PCR (dPCR) is employed to quantify the amount of target nucleic acid in a sample. Both technologies are capable of detecting as little as 1 target copy within a sample containing numerous sequences that are not of interest. To quantify using qPCR requires a standard curve to be created from samples containing known amounts of target nucleic acid copies of interest. Once the curve is established, the concentration of target nucleic acid sequences can be extrapolated based on the Ct (threshold cycle) value derived from each experimental sample. Digital PCR allows for direct quantitation of target nucleic acid sequences by counting partitions that are determined to be positive. Often this will require a correction for possible multiple occupancy of targets of interest within a partition. While dPCR allows for absolute quantitation, it does not provide the same dynamic range of qPCR. Direct quantitation, dynamic range and cost are the primary differences between qPCR and dPCR for target nucleic acid quantification.

In some embodiments, an oligonucleotide linked to the detection agent comprises one or more primer binding sites, e.g., for an amplification reaction. The amplification reactions can be any amplification reaction. In some embodiments, the amplification reaction is a quantitative PCR reaction. Alternative amplification reactions to determine positive pools include T7 amplification, rolling circle amplification (RCA), loop-mediated isothermal amplification (LAMP) or any other suitable amplification reaction. For example, LAMP or RCA amplification reactions can be employed to generate a fluorescently amplified product that can be quantified.

In some embodiments, the oligonucleotide hybridizes to a complementary oligonucleotide attached to a bead, e.g., to facilitate sequence analysis, or an alternative surface, e.g., for image analysis.

### Fast Off-Rate Binding Agents

Fast off-rate binding agents that specifically bind an analyte of interest are used herein as capture agents to capture analyte-detection agent complexes as they migrate through a channel, thereby delaying the mobility of the complex compared to un-complexed detection agents. It will be apparent to those of skill in the art that the equation K_{d}=k_{off}/kₒₙ characterizes transient binding under fast-off rate conditions.

A fast-off-rate binding agent, e.g., a Fab, that binds to the target analyte typically has a kinetic rate constant k_{off} that is about 0.1 sec⁻¹ and/or a kinetic rate constant kₒₙ that is greater than 1×10⁵M⁻¹ sec⁻¹. For example, in some embodiments, the kinetic rate constant kₒₙ describing the association of the capture agent with analyte is greater than 1×10⁵ M⁻¹ sec⁻¹, or 1×10⁶ M⁻¹ sec⁻¹, or higher. Most often the upper limit to kₒₙ is related to the diffusion coefficient and could approach 1×10⁸ M⁻¹ sec⁻¹-1×10⁹ M⁻¹ sec⁻¹. In some embodiments the kinetic rate constant k_{off} describing the dissociation of the complex is/are greater than 0.01 sec⁻¹, e.g., greater than 0.1 sec⁻¹ or greater than 1 sec⁻¹ or greater than 10 sec⁻¹. In some embodiments, the kinetic rate constant k_{off} describing the dissociation of the complex is/are great than 0.01 s⁻¹, e.g., great than 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10 s⁻¹ or greater. In some embodiments, on-rates for a such a binding agent may be 10⁵ or 10⁶ M⁻¹ s⁻¹. In some embodiments, the K_{D} of a binding agent is from about 10 nM to about 10 μM.

### Solid Supports

In one embodiment, the fast off-rate capture agent (e.g., a Fab) is immobilized on a solid support surface. Such surfaces include, but are not limited to, a channel, nanoparticle, a microsphere, bead, or a polymer matrix. In typical embodiments, capture agents are immobilized onto the surface of beads or microparticles that are packed into a channel, or immobilized onto a polymer matrix such as agarose or polyacrylamide, e.g., a linear or cross-linked polymer that allows passage of large molecules.

A “solid support” refers to a material or group of materials having a rigid or semi-rigid surface or surfaces. In some embodiments, the solid support is matrix, such as a hydrophilic polymer, e.g., a polymer that is insoluble and swells in water. Suitable polymers include, but are not limited to polyhydroxy polymers, e.g., based on polysaccharides, such as agarose, dextran, cellulose, starch, pullulan, etc. and completely synthetic polymers, such as polyacrylic amide, polymethacrylic amide, poly(N,N-dimethylacrylamide, poly(hydroxyalkylvinyl ethers), poly(hydroxyalkylacrylates) and polymethacrylates (e.g. polyglycidylmethacrylate), polyethylene glycol polymers, polyvinyl alcohols and polymers based on styrenes and divinylbenzenes, and copolymers in which two or more of the monomers corresponding to the above-mentioned polymers are included. Polymers, which are soluble in water, may be derivatized to become insoluble, e.g. by cross-linking and by coupling to an insoluble body via adsorption or covalent binding. Hydrophilic groups can be introduced on hydrophobic polymers (e.g. on copolymers of monovinyl and divinylbenzenes) by polymerization of monomers exhibiting groups which can be converted to OH, or by hydrophilization of the final polymer, e.g. by adsorption of suitable compounds, such as hydrophilic polymers. In some embodiments, the support is an UNOsphere™ support, a polymer produced from water-soluble hydrophilic monomers (Bio-Rad, Hercules, CA). Alternatively, the matrix is agarose (GE Sepharose or Sterogene Superflow and Ultraflow). In some embodiments, the support is comprised of glass or synthetic fibers that have been activated with chemical groups that react with protein or nucleic acids such as Fabs or aptamers with fast off-rates. In still other embodiments, the support is a membrane material, such as nitrocellulose that can non-covalently bind the capture agents having fast off-rates as described in the section detailing lateral flow assay embodiments of the invention.

In the present disclosure, analyte-detection agent complexes are allowed to contact a surface with immobilized capture molecules and interact transiently with fast-off rate binding agents. The transient binding events slow migration across or through the surface or matrix. In some embodiments, a population of capture agents comprising a heterogeneous mixture of capture agents to the analyte of interest is immobilized on the solid support. In some embodiments, a homogeneous population of capture agents is immobilized to the solid support. In some embodiments, a population of capture agents bound to a solid support comprises different types of binding agents, e.g., a mixture of Fabs, aptamers, ligands, or other agents that have fast-off rates binding kinetics for the target analyte. In some embodiments the solid support comprises regions having different capture agents that are specific to different target analytes.

Capture agents that bind to analyte with a fast off-rate can be identified using any number of methods. In some embodiments, such a binding agent is selected from random libraries of peptides, small ligands, small molecules, aptamers and the like. In some instances, capture agents, e.g., Fabs, that bind the analyte of interest are generated using combinatorial techniques and library screening, e.g., phage or yeast display library screening, well known to those of skill in the art. For example, Fabs can be generated using the HuCal technology from Bio-Rad Laboratories. In addition, capture agents can be formed by synthetic molecules. (Iterative In Situ Click Chemistry Creates Antibody-like Protein-Capture Agents, H. D. Agnew et al., Angew. Chem. Int. Ed. 2009, 48, 4944-4948.) (Accurate MALDI-TOF/TOF Sequencing of One-Bead-One-Compound Peptide Libraries with Application to the Identification of Multiligand Protein Affinity Agents Using in Situ Click Chemistry Screening, Su Seongi Lee et al., Anal. Chem., 2010, 82 (2), pp 672-679.)

Many different techniques can be used to immobilize capture agents to a solid support. In some embodiments, immobilized carboxylate groups on an amine-reactive surface can be used to covalently link polypeptide capture agents to the substrate via an amine-coupling reaction. Other illustrative reactive linking groups, e.g., hydrazines, hydroxylamines, thiols, carboxylic acids, epoxides, trialkoxysilanes, dialkoxysilanes, and chlorosilanes, may be attached to the substrate, such that polypeptides can form chemical bonds with those linking groups to immobilize them on the substrate. In some embodiments the capture agents can be copolymerized into the solid support by first adding the appropriate chemical group such as an acrylic group to the capture agent. In some embodiments the capture agents can be attached using click chemistry. In some embodiments the capture agents are bound non-covalently to a support, such as to nitrocellulose membranes, following deposition of the agents onto the support.

In some instances, a polypeptide can be immobilized on a surface using methods such as a hydrophilic self-assembled monolayer approach, a hydrophilic polymer brush approach, a zwiterionic polymer brush approach and a nitrile coating approach.

In some embodiments, the surface may also be coated with streptavidin at a known concentration, followed by attachment of biotin labeled binding agent. In still other embodiments the capture agents can be immobilized using other bridging molecules that form non-covalent or covalent complexes such as Spytag and Spycatcher complexes where one component of the system could be present on the capture agent and the other on the solid support.

### Migration of Detection Agent-Analyte Complex

Various parameters can influence the rate of migration of a detection agent-analyte complex through a channel or compartment. Such parameters include the pH of the solution, the density of capture agents immobilized to the solid support, the size of the channel, e.g., diameter and length, flow rate through the channel, or other force applied such as electric field strength.

In some embodiments, capture agents, e.g., Fabs, are immobilized onto a solid support at a high density, e.g., a density of at least 10,000 molecules per μm² of surface area or at least 20,000 molecules per μm² of surface area; or about 50,000 molecules or greater per μm² of surface area. In some embodiments, a resin is prepared using from 1 μg to 1 mg of capture agent. As understood by one of skill in the art, the density of capture agents is adjusted to provide sufficient transient interactions of the analyte-detection agent complex with the solid support such that the complex is temporally and spatially resolved from non-specific interactions of the un-bound detection agent with the same solid support.

In some embodiments, the diameter of the channel is about 20 μm to about 500 μm, or in some embodiments from about 50 μm to about 200 μm. In some embodiments, the channel is about 100 μm or less in diameter, e.g., from about 1 μm to about 10 μm, or to about 100 μm, in diameter. In some embodiments, the length of the channel is proportional to the volume of the sample, e.g., about 20, about 50, or about 100 times the volume of the sample.

In some embodiments, the channel is the channel of a microfluidic device. Thus, in some embodiments, an initial eluate flowing through the channel can be diverted into another channel, thus providing a solution comprising detection agent/analyte complex that is free of unbound detection agent.

In some embodiments, migration through the channel or compartment, is modulated by manipulating various parameters that influence the affinity of the capture agent for binding to target analyte present in the detection agent-analyte complex. In some embodiments, an electric field or pressure or centrifugal force may be applied to modulate migration through the channel. In some embodiments, as noted above, temperature, pH, and/or ionic composition of the solution comprising the detection agent-analyte complex is manipulated to modulate flow of the complex through the channel or compartment. In some embodiments, one or more wash steps may be employed to further remove or resolve free detection agent from the detection agent present in the analyte-detection agent complex. One of skill in the art understands how to adjust such parameters to achieve a desired rate of flow through the channel or compartment.

In some embodiments, the detection agent/analyte complex in the eluate is concentrated using beads, magnetic beads with complementary nucleic acid, chromatography resins (e.g. IEX, hydrophobic interaction), filter or membrane concentrators, or another method known in the art to concentrate proteins and/or nucleic acids. In some embodiments, the detection agent/analyte complex is recovered from a polymer support via application of an electric field, pressure, or other force in an orthogonal direction. In some embodiments, the detection agent/analyte complex is recovered from the polymer support via excision of an area containing the detection agent/analyte complex.

An eluate obtained comprising the detection agent/analyte complex following migration through the channel or compartment is substantially free of unbound detection agent. A blank sample without analyte is employed to subtract background signal from un-complexed detection agent to determine the limit of sensitivity. Analyte-containing samples preferably exhibit a Signal:Noise ration of >3 as the threshold for the limit of detection.

### Lateral Flow Assay

Lateral flow configurations that allow for sequential flow of reagents through a wicking matrix are known, e.g., U.S. Pat. Nos. 10,688,487; 10,883,987; and 10,591,477. In embodiments of the assay of the invention that employ lateral flow, the matrix itself with bound capture agent, e.g., a Fab, would afford the separation with the lateral flow being used to create a flow path and control the delivery of solutions. Reagents are supplied to the matrix in sequential fashion (see, e.g., U.S. Pat. No. 10,883,987). The surface employed in the flow assay may be a membrane such as a nitrocellulose membrane, e.g., having a porosity of 0.1 μm, 0.2 μm, 0.45 μm, 1 μm, 3 μm, 5 μm, 8 μm, or 10 μm. In some embodiments, the capture agent, e.g., a fast off-rate Fab is deposited and binds to a discrete region. The surface may then be blocked (e.g., with bovine serum albumin or alternative blocking agent) and washed. In some embodiments, the lateral flow surface may be glass fiber, cellulose, or synthetic fiber or a combination thereof. In some embodiments the flow surface is polyvinylidene fluoride, nylon or polysulfone membrane. In such instances, the capture agent, e.g. fast off-rate Fabs, are covalently and/or non-covalently attached to the surface using chemical/biochemical means or through linker molecules such as streptavidin and biotin among others, as detailed above in the “Solid Support” section.

The flow paths in the membrane/wicking matrix can be delineated using hydrophobic or physical barriers using wax, acrylic, and the like that can fill the pores of the matrix creating a wall to define the flow paths (see, e.g., U.S. Pat. No. 10,883,987). A commercial example of delineated flow paths formed in nitrocellulose membranes are the Unisart StructSure® Membranes products from Sartorius. In preferred embodiments, the dimensions of the flow path are microfluidic, or a few mm in width and depth and cm in length to allow sufficient flow distance to afford the separation as needed.

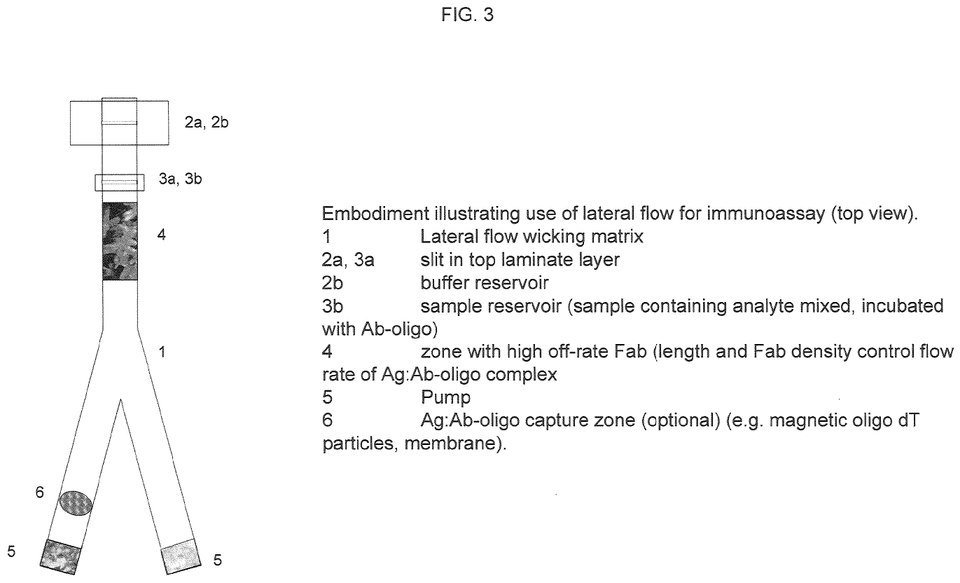

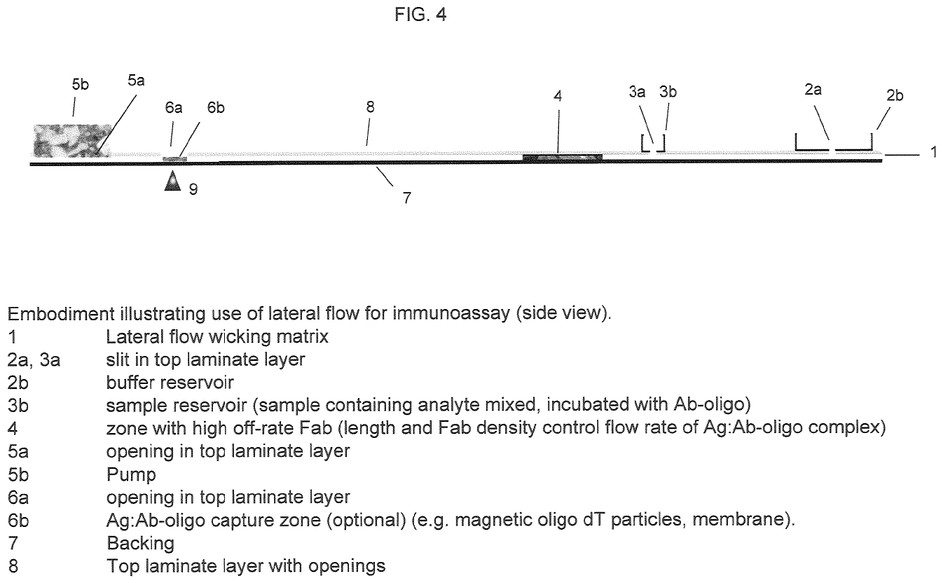

In some embodiments, the assay is designed to employ a Y or double Y flow path, e.g., to minimize carryover of free detection agent, e.g., oligonucleotide-conjugated high affinity antibody, into the final eluate. FIGS. 3 and 4 provide top and side view illustrations of one embodiment using lateral flow. This illustrative embodiment contains a Y split at the end and shows a zone where the Ag:Ab-oligo complex can be captured.

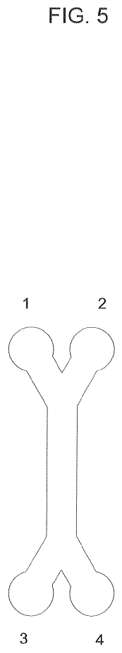

FIG. 5 illustrates a double Y flow path. Thus, for example, with this design, a sample can be applied to port 1, and the separation proceed along flow path to port 4 for a prescribed period to allow free detection agent, e.g., oligonucleotide-conjugated detection antibody (abbreviated Ag:Ab-oligo complex for purposes of this example), to be eluted to port 4 The flow path can then be changed along flow path 2 and 3 to effect elution of the detection agent-analyte complex, e.g. Ag:Ab-oligo complex, to port 3 for further analysis, e.g., by PCR.

Other configurations of split flow paths that achieve the desired goal of reduced background, optimal flow and analysis can be envisioned where the number and angle of the arm positions are different, such as where the arm(s) intersect the initial path at 90 degrees. An optional concentration step, e.g., binding to a membrane, magnetic particles, and the like, may also be incorporated before further analysis.

### Samples

A sample can be any composition containing an analyte of interest, including, for example, a food, soil, or water sample, or a biological sample obtained from an organism. In some embodiments, the analyte is a protein or a plurality of proteins. Such proteins include, but are not limited to, polypeptides, small peptides, glycoprotein, lipoproteins antibodies, enzymes, disease markers (such as polypeptide cancer antigens), cell surface receptors, hormone receptors, cytokines, chemokines, tissue specific antigens, or fragments of any of the foregoing. In some embodiments, the sample can be a microorganism, e.g., a virus.

In some embodiments, the sample is a biological sample. Biological samples can be obtained from any organism, including, for example, animals, plant, fungi, bacteria, protozoa, or viruses. In some embodiments, the sample, is a bodily fluid or excretion, e.g., a blood, serum, plasma, urine, cerebrospinal fluid, cell secretion, saliva, sputum, or stool sample. In some embodiments, the biological sample is a cell or tissue, e.g., a tissue sample from an organ, a biopsy, an explant, or preserved or fixed tissue samples; or a cellular sample, including cancer cells, cultured cells, or preserved or fixed cells. In some embodiments, the biological sample is a supernatant from a cellular preparation, for example, medium in which a cell is cultured.

In some embodiments, assays are performed as multiplex assays to detect more than one analyte in a sample; and/or to evaluate multiple samples at the same time. One of skill understands that detection agents for different target analytes and/or samples can be labeled with detectable labels that can be distinguished from one another, e.g., barcoded oligonucleotides or different fluorescent labels.

### Kits

In some embodiments, a solid support with the population of low affinity Fabs that bind target analyte is packaged in a kit. In some embodiments the solid support comprises 96-well, 384-well, or more wells in a plate format for ease of automated processing and to align to methods used for the generation of and measurement of the signal associated with the detection agent. In some embodiments, the solid support to which Fabs are immobilized are microbeads, e.g., which may be contained in a channel or container. In some embodiments low affinity Fabs are immobilized to a membrane, e.g., nitrocellulose. In some embodiments, the kit further comprises a high affinity detection agent; and/or reagents e.g., oligonucleotide primers, probes, or other reagents generate a signal from the detection component of the detection agent.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of quantifying a target analyte, the method comprising
incubating (i) a solution comprising a complex that comprises a target analyte bound to a detection agent, wherein the detection agent binds with high affinity to the target analyte and is labeled with a detectable label; and (ii) a population of capture agents immobilized to a solid support in a channel or compartment or immobilized in a discrete zone on a solid support; wherein the capture agents specifically bind the analyte and have a high off-rate of binding to the target analyte, and the analyte:detection agent complex migrates across the channel or compartment; or across the discrete zone;
spatially resolving the complex from other molecules in the solution based on rate of migration of the complex across the solid support relative to unbound detection agent to provide an eluate comprising the complex substantially free of unbound detection agent; and
quantifying the amount of detection agent complexed with analyte, thereby determining the amount of the target analyte present in the solution.

2. The method of claim 1, wherein the capture agents are Fabs or aptamers.

3. The method of claim 1, wherein the high affinity detection agent is an aptamer, antibody, or ligand.

4. The method of claim 3, wherein the high affinity detection agent is an antibody.

5. The method of claim 1, wherein the detectable label is specific for the analyte.

6. The method of claim 1, wherein the detectable label is an oligonucleotide.

7. The method of claim 6, wherein the oligonucleotide comprises a primer binding site or comprises a sequence that targets a primer-binding site of a detection oligonucleotide that is detected in an amplification reaction; and/or the oligonucleotide comprises an analyte identification region and/or a sample identification region.

8. The method of claim 6, wherein quantifying comprises amplifying a target region of the oligonucleotide to obtain an amplicon or quantifying comprises sequencing a region of the oligonucleotide specific for the analyte to determine the amount of oligonucleotide present in the detection agent-analyte complex.

9. The method of claim 8, wherein amplifying a target region of the oligonucleotide to obtain an amplicon comprises a PCR.

10. The method of claim 9, wherein the PCR is a quantitative PCR.

11. The method of claim 1, wherein the complex comprises a fluorescent label.

12. The method of claim 11, wherein quantifying comprises detecting the level of fluorescent signal generated by the fluorescent label.

13. The method of claim 1, wherein the channel is a capillary.

14. The method of claim 1, wherein the solid support is a plurality of beads present in the channel or compartment.

15. The method of claim 1, wherein the channel or compartment contains a solid support comprised of a polymer.

16. The method of claim 1, wherein pressure or an electric field is applied to the solution as it flows through the channel.

17. The method of claim 1, wherein the channel is a channel of a microfluidic device or the channel is a capillary present in a microwell.

18. The method of claim 1, wherein the solid support is a membrane or wicking matrix and the solution flows through the membrane or wicking matrix by lateral flow.

19. The method of claim 18, wherein the solid support is nitrocellulose.

20. A kit comprising a capture agent bound to a solid support and a high affinity antibody.

21. The kit of claim 20, wherein the solid support is a microbead or microparticle.

22. The kit of claim 20, wherein the solid support, is a membrane or wicking matrix.
